(19) United States
(12) Patent Application Publication
(10) Pub. No.: US 2015/0266781 A1
(43) Pub. Date: Sep. 24, 2015

(54) CERAMIC AND METHOD FOR THE PRODUCTION THEREOF

(71) Applicant: CERAMTEC GMBH, Plochingen (DE)

(72) Inventors: Heinrich Wecker, Eckental (DE); Uwe Kemmer, Wernau (DE)

(21) Appl. No.: 14/435,963

(22) PCT Filed: Oct. 15, 2013
(86) PCT No.: PCT/EP2013/071458

§ 371 Date: Apr. 27, 2015

(30) Foreign Application Priority Data
Oct. 16, 2012 (DE) 10 2012 218 796.0
Oct. 25, 2012 (DE) 10 2012 219 492.4

(51) Int. Cl. C04B 35/80; C04B 35/71; A61F 2/30; C04B 35/64

(52) U.S. Cl. CPC C04B 35/80; C04B 35/64; C04B 35/71; A61F 2/30

(57) ABSTRACT

A ceramic and a method for the production thereof. The ceramic has a graded increase of reinforcing elements in at least one direction.

The invention relates to a ceramic and a method for the production thereof. The invention relates in particular to a ceramic composite material, which has a graded increase of reinforcing elements in at least one direction.

Ceramics with and without reinforcing elements are known from the prior art. Ceramics without reinforcing elements are used, for example, as part of implants. The disadvantage here is usually that the ceramic must be combined with metallic parts, such as in the case of a ceramic acetabulum inlay, which is inserted intra-operatively in a metal socket, often made of titanium. The metal parts serve for anchoring in the bone, where they are exposed to dynamic loading. However, ceramic materials may be exposed to punctual stress in direct contact with the metal. Dynamic loadings, in particular in the case of point loading acting on the ceramic, can even result in breakage of the ceramic.

However, ceramics possess very great advantages, in particular in joints, on the basis of their tribological properties. They can be polished very smooth, so that in the case of ceramic-ceramic-pairings only minor frictional torques occur. Moreover, ceramics can be very hard, so that only slight wear occurs. In addition, abrasion debris of bio-inert ceramics is not toxic in contrast to metal abrasion debris.

The combination metal/ceramic is not without problems, however, precisely because the materials have very different properties. Dynamic loadings can result in breakage of ceramics due to the comparatively low flexural strength thereof. Especially the area which is in direct contact with the metal component, is subject to particular loadings through the transfer of forces, and thus, punctual stresses on these interfaces should be avoided.

Therefore, the object of the invention is to provide a ceramic body having a high tolerance with respect to punctual stresses, and to provide a method of production for such a ceramic compound.

The object is achieved with the features of claims 1 and 8. Accordingly, a ceramic body according to the invention comprises a ceramic matrix and reinforcing elements embedded therein, whereby the ceramic matrix has a graded texture in at least one direction. The term “in one direction” comprises in particular the embodiment that the gradation extends in a direction of at least one surface of the ceramic compound.

According to different exemplary embodiments of the invention, the graded texture can be formed by the amount of reinforcing elements, the size of the reinforcing elements and/or type of the reinforcing elements. In particular, reinforcing elements which can be used are ceramic elements such as fibers, whiskers or platelets. Particularly preferred as reinforcing elements are ceramic short fibers, in particular coated ceramic short fibers.

The concept of gradation of composite materials leads to a continuous variation of all important characteristic values, such as modulus of elasticity, fracture toughness and strength. With respect to mechanical properties, gradients enable optimal adaptation to external requirements of a progression of properties in materials. However, graded components may also have functional properties that cannot be achieved by a direct material transition.

Advantageously, oxide ceramic materials can be used. Oxide ceramic materials display high thermal and chemical stability in comparison to metallic materials or plastics. A quasi-ductile deformation behavior can be achieved by the integration of high-strength ceramic fibers. The fiber/matrix bonding has a decisive influence on a quasi-ductile, damage-tolerant deformation behavior, wherein mechanisms such as crack deflection, crack branching can take effect. The bonding between fibers and matrix can be specifically adjusted owing to the properties of the ceramic matrix itself and/or suitable fiber coatings.

For instance, if the matrix is relatively porous and less dense, then the coupling to the fibers is weak. Damages and cracks are then directed through the matrix and deflected from the fibers. For example, if the matrix is very dense and thereby the coupling to the fibers is high, cracks can also run through the fibers. Depending on the fiber coating it can be determine whether the fibers detach from the matrix (“pull-out”) or remain in the matrix. The possibility is thus opened to set and adjust the failure behavior optimally for the intended use.

The production of the ceramic composite is relatively simple and economical.

Preferably, the amount of reinforcing elements can be increased in the direction of at least one surface of the ceramic compound. Thus, for example, the contact area between ceramic and metal can be adapted to the requirements by use of the reinforcing elements, particularly preferably by use of ceramic fibers. Ceramic fibers can alter the E-modulus of the ceramic. An altered E-modulus in the contact area between ceramic and metal advantageously improves the flexural strength of the ceramic and thereby the resisting force thereof against dynamic loadings. The concurrently decreasing hardness of the material can thereby be limited to the contact area to the metal, so that the good, in particular tribological properties with respect to the joint surfaces are still retained. The gradation of the reinforcing elements serves, inter alia, to avoid abrupt transitions, which on their part could in turn negatively influence the susceptibility to breakage of the ceramic.

Thus, a preferred embodiment of the invention provides a ceramic body, in which at least one surface is or can be connected non-positively to a metallic component, and wherein the reinforcing elements in the ceramic compound continuously increase in the direction of the surface having the non-positive connection. Thus, a graded texture is created, wherein the amount and/or the size and/or the type of reinforcing elements increases in the direction of the surface having the non-positive connection.

According to a further embodiment of the invention, the ceramic body may comprise a core that is substantially free of reinforcing elements, while the amount of reinforcing elements increases toward the surface or surfaces of the ceramic body.

A ceramic body according to the invention can be produced with various methods of ceramic shaping. These are all established methods known in principle to the person skilled in the art, but which still must be adapted to the ceramic body according to the invention.

A ceramic body according to the invention can be produced, for example, by means of ceramic injection molding (CIM), with which a close-contour shaping of the ceramic body can be carried out. To this end, the short fibers can be injected into the mold using a single-stage or multi-stage process.

Another production method is based on a molding technique, in which the fibers can be introduced oriented, for example via mechanical means, into a casting slurry.

Another production method is based on an infiltration technique, in which for example, the prefabricated nonwovens (short fibers), scrims or fabrics (long fibers) are soaked with ceramic slurry. Particular embodiments for this purpose are freeze casting or gel casting, for example.

The thermal processes for debindering and sintering which follow the shaping in the green state of the ceramic body according to the invention likewise still represent a great challenge, in particular because thereby the matrix-fiber coupling and the microstructure of the ceramic composite material is substantially influenced. But here too, techniques are used which are known in principle to the person skilled in the art.

The hard machining and/or finishing treatment of the ceramic body is likewise based on known techniques, wherein the specific properties of the ceramic composite are also to be taken into account.

The ceramic bodies described above can be used in medical technology, for example, as part of an exoprosthesis, endoprosthesis, such as a knee-, hip-, shoulder- or vertebral column implant, trauma nail, bone screw or trauma plate. In principle, they may be used for all types of implants (temporary, long-term, biocompatible, invasive, etc.) and for instruments or instrument parts in particular in medical technology.

Moreover, such a ceramic body can be used as a component of a tool, in particular as part of a medical tool.

A ceramic body of the type described can also be used wherever a quasi-ductile deformation behavior with fracture toughness >15 MPa√m, as is customary with metals (CoCr), is required.

Due to the good tribological properties of ceramics, a further field of application is a field where tribological aspects play a role and a hardness of at least 17 GPa/HV1 should be provided.

Further fields of application, based on the wide variety of possible combinations of properties and structural design, are all fields in which
failure due to static and/or dynamic loading should be avoided,
failure due to abrasion/loading due to daily operation should be avoided,
failure due to abrasion/loading due to operative constraints should be avoided,
interface between metal and ceramic should be improved,
force transmission elements are necessary.

1.-12. (canceled)

13. A ceramic body comprising:
a ceramic matrix; and
reinforcing elements embedded in the ceramic matrix;
wherein the ceramic body has a graded texture in at least one direction.

14. The ceramic body according to claim 13, wherein the amount of reinforcing elements, the size of the reinforcing elements and/or the type of the reinforcing elements varies.

15. The ceramic body according to claim 13, wherein the reinforcing elements comprise ceramic elements.

16. The ceramic body according to claim 15, wherein the reinforcing elements comprise coated ceramic short fibers.

17. The ceramic body according to claim 13, wherein at least one of the amount, the size or the type of reinforcing elements increases in the direction of at least one surface of the ceramic body.

18. The ceramic body according to claim 17, wherein the at least one surface can be connected or is connected in a nonpositive manner to a metallic component.

19. The ceramic body according to claim 13, wherein the ceramic body comprises a core, and wherein said core is substantially free of reinforcing elements or comprises less reinforcing elements, while the amount of reinforcing elements increases toward at least one of the surfaces of the ceramic body.

20. A method for the production of a ceramic body having a graded texture.

21. The method for the production of a ceramic body according to claim 20, wherein the method comprises at least the steps of introducing reinforcing elements into a slurry, shaping and sintering.

22. The method according to claim 20, wherein the method comprises ceramic injection molding (CIM) or a casting technique or an infiltration technique, in particular freeze casting or gel casting.

23. An exoprosthesis or endoprosthesis comprising the ceramic body of claim 13.

24. An exoprosthesis or endoprosthesis according to claim 23, wherein the endoprosthesis or endoprosthesis is in a form selected from the group consisting of a knee joint implant, a hip joint implant, a shoulder implant a vertebral column implant, a trauma nail, a bone screw and a trauma plate.

25. A medical tool or a medical instrument comprising the ceramic body according to claim 13.

26. The ceramic body according to claim 14, wherein as reinforcing elements, ceramic elements, in particular fibers, whiskers or platelets, are comprised.

27. The ceramic body according to claim 14, wherein the reinforcing elements comprise ceramic short fibers, in particular coated ceramic short fibers, are comprised.

28. The ceramic body according to claim 14, wherein the amount and/or the size and/or the type of reinforcing elements increases in the direction of at least one surface of the ceramic body.

29. The ceramic body according to claim 13, wherein the ceramic short fibers are coated ceramic short fibers.

30. The ceramic body according to claim 13, wherein at least one of the amount increases in the direction of at least one surface of the ceramic body.

31. The ceramic body according to claim 13, wherein at least one of the size of reinforcing elements increases in the direction of at least one surface of the ceramic body.

32. The ceramic body according to claim 13, wherein at least one of the type of reinforcing elements increases in the direction of at least one surface of the ceramic body.
